(19) United States
(12) Patent Application Publication
(10) Pub. No.: US 2011/0251625 A1
(43) Pub. Date: Oct. 13, 2011

(54) MEDICAL NAVIGATION SYSTEM AND METHOD FOR THE OPERATION THEREOF

(76) Inventors: Clemens Bulitta, Spardorf (DE); Tim Dannenmann, Bamberg (DE); Rainer Graumann, Hochstadt (DE); Markus Nagel, Nurnberg (DE); Martin Ringholz, Erlangen (DE)

(21) Appl. No.: 13/127,939

(22) PCT Filed: Oct. 28, 2009
(86) PCT No.: PCT/EP09/64171

§ 371 Date: May 5, 2011

(30) Foreign Application Priority Data
Nov. 5, 2008 (DE) 10 2008 055 918.0

(51) Int. Cl. A61B 19/00

(52) U.S. Cl. 606/130

(57) ABSTRACT

In a medical navigation system and an operating method therefor, a navigation device, having a field of view, detects a position indicator attached to a subject within the navigation field of view, and the navigation device, and thus the navigation field of view, are varied in position by a motorized drive connected to the navigation device. The current attitude of the navigation device and the position indicator are detected, and based on this detection, the motorized drive is operated and controlled so as to always maintain the position indicator within the navigation field of view of the navigation device.

### BACKGROUND OF THE INVENTION

1. Field of the Invention

The invention concerns a method to operate a medical navigation system, and a medical navigation system.

2. Description of the Prior Art

For medical procedures on patients—for example diagnoses, therapies or operative procedures—a spatial accuracy that is as high as possible is desirable. In surgical procedures—for example on the brain or the spinal column of a patient—it is even indispensable to operate on a precisely determined location in the patient with a medical instrument. Systems known as medical navigation or tracking systems for this purpose have existed for a long time. Systems that operate optically or electromagnetically are significantly different. A medical navigation system operates such that a location indicator in the form of a marker is attached to each of the patient and to the surgical instruments to be used. This marker—a trihedron made of spheres that can be easily detected optically in the optical case, for example; a sensor coil in the electromagnetic case—interacts with a navigation device (mounted so as to be stationary) of the navigation system, in the optical case one or more cameras and in the magnetic case one or more field coils. In the optical case, the camera detects the spatial position of the marker; in the electromagnetic case the spatial position is determined by evaluating the field of the field coil that is received in the sensor coil. In ceiling-based navigation systems, the navigation device is attached to the room ceiling, for example of the operating room. Mobile systems contain a camera mounted on a stand as a navigation device which can be placed arbitrarily freely in space, for example in the operating room.

Such a navigation device covers only a certain spatial volume (known as the tracking volume) as a navigation region, which the position of the position indicator can be detected only by this navigation region. In particular in optical navigation systems, the optical camera systems must be readjusted given a change of the OP setup if the camera no longer has a line of sight to markers on instruments or patient. In the case of an optical occlusion—for example as a result of a relocation of the patient—the user must thus manually reposition the optical tracking system, i.e. shift the camera arranged on a stand or on the OP room floor, for example, in order to ensure sufficient visibility of the position indicator in the region of interest (for example the situs). In electromagnetic navigation systems, the field generator merely generates a magnetic field in a limited spatial region, in which magnetic field the receiver coils function with sufficient precision. The field generator must then be occasionally displaced.

With regard to the OP workflow, such adjustments are not desired since effort is necessary and time delays result; the OP workflow is disrupted. For example, the tracking is stopped as soon as there is no line of sight. If the position indicator no longer lies in the tracking volume, the current location of a surgical instrument is temporarily unknown to the navigation system. Without the position information, the medical procedure must be interrupted. For example, in procedures on the spinal column of a patient in proximity to the spinal cord this is problematic since here unintended injuries can lead to complications for the patient. Given known navigation systems, the OP personnel therefore specifically ensure that in every conceivable situation of the procedure the position indicators are situated in the navigation region, or ensure that the navigation device generates a suitable navigation region and the navigation region is not disrupted by the OP setup in any situation of the procedure.

In known systems—thus in the aforementioned cases—the navigation device must thus be readjusted manually, for example by a physician or OP personnel. In a sterile OP environment it is an additional complication that maintaining a sterile environment must be taken into account. The navigation system is normally unsterile and is provided with appropriate sterile coverings or wrapping that must be displaced as well given tracking of the system. Manual contact with the unsterile navigation system is particularly problematical.

### SUMMARY OF THE INVENTION

An object of the invention is to provide an improved method to operate a medical navigation system and an improved medical navigation system.

The above object is achieved in accordance with the invention by a method to operate a medical navigation system, wherein the navigation system has a navigation device that is associated with a limited navigation region. The navigation system moreover has at least one position indicator that can be attached to a subject to be located and that can be located only within the navigation region. The navigation system has a motorized drive for position and attitude variation of the navigation device. By the spatial variation of the navigation device, the attitude and alignment of the navigation region ultimately coupled with it are also varied. The navigation system also has a detection device to detect the current attitude and alignment of navigation region and position indicator. According to the invention, the navigation system detects the attitude variation of the navigation device that is implemented by the motorized drive. An ongoing regulation of the coordinates of the attitude variation additionally controls the drive via a control signal such that the navigation region contains always the position indicator at any given time. For this purpose, the regulation uses an input signal from the detection device that provides information about the current attitude of the navigation region of the position indicator.

The invention is based on the insight to always move the navigation device via a motorized drive and a correspondingly suitable controller or regulator, to cause the tracking volume—and thus the navigation region—to always be aligned on the presently required spatial region at any time. The spatial region is thus always automatically held or selected by the controller so that the necessary position indicator (for example of the medical instrument) and the patient reference (thus the position indicator attached to the patient) are always located inside said spatial region.

In other words, the navigation system or its navigation device can be optimally aligned by the integrated regulator even when instruments with corresponding position indicators are held or moved at disadvantageous angles or distances relative to the navigation device, for example. The regulator thus always seeks an optimal or improved position as long as the position indicator by varying the navigation region, can be better placed in this improved position. A manual readjustment of the navigation device—for example by OP personnel—is no longer necessary. The user of the navigation system loses no time and in general does not have to worry about the suitable placement of the navigation device, for example does not need to think about the setting of optimal visibility conditions for a navigation camera. The user does not need to make himself or herself unsterile due to contact with components of the navigation system and loses no time with work that is inconvenient for the user. The navigation is more comfortable to use due to this method and thus its acceptance is increased. The risk to the patient is markedly reduced.

In a preferred embodiment of the invention, the regulator activates the drive such that the navigation device has a defined distance from the position indicator. For example, an optimal distance between camera and marker or field generator and receiver coil is preselected (thus defined) for a given navigation system, and the navigation device is held at a correspondingly optimal distance by the controller.

The medical navigation system normally has at least two position indicators. In a preferred embodiment of the method, the regulator controls the drive such that the navigation device is centered with regard to the position indicator. In other words, the navigation device passes to a position that, for example, enables an average, optimally identical distance from the different position indicators or the position distances between navigation device and the position indicators exhibit an optimally small fluctuation range around an optimal distance.

In a further preferred embodiment of the method, the navigation device operates optically and operates simultaneously as a detection device. In other words, for example, a tracking camera is used simultaneously for the actual navigation but also to detect the current navigation region, even the viewing angle or field of view of the navigation camera. An additional, separate detection device is accordingly superfluous.

In an alternative method variant, the navigation device operates electromagnetically and the position indicator simultaneously operates as a detection device. In other words, here a sensor coil is us used both for navigation and to measure the electromagnetic field generated by the navigation device in order to thus determine the navigation region. An additional separate detection device is likewise not necessary here.

In the case of an electromagnetic navigation device, in a further embodiment of the method the regulator also activates the drive such that the electromagnetic field generated by the navigation device at the location of the position indicator has a specific alignment. For example, the controller can always align the field coil such that the generated field at the location of the sensor coil always has optimal field alignment, for example it is perpendicular to this.

In a further embodiment of the method, the detection device monitors the navigation region for interfering foreign objects. In the case of a tracking camera, for example, the detection device monitors its field of view for the penetration of interfering foreign bodies (i.e. foreign bodies that occlude the camera's view), for example OP personnel or a voluminous instrument (for example an x-ray C-arm). The spatial position of the corresponding foreign objects can then be detected, and the regulator can determine a new position for the navigation device so that again there is free view of the position indicator.

In a further embodiment of the method, the detection device monitors the navigation region with a camera coupled with an image processing system. A monitoring of the navigation region for foreign objects thus is possible in a particularly simple manner.

In an embodiment of the method, additional knowledge or additional data is or are detected via redundant or additional components of the detection device. For example, a larger navigation region can be tracked via a second camera. If a camera becomes “blind” due to occlusion, the second (thus redundant) camera delivers additional tracking information. At this time the occluded system—thus the first camera—serves for a new, optimal position. The controller then uses these data or, respectively, the knowledge for the optimization of the position of the navigation device. Redundant or additional components are, for example, a second optical tracking system or a video camera with connected image processing that can acquire and evaluate the image information with regard to movements, position displacements of components of the OP setup, etc. For optical navigation systems, such a second optical tracking system could for example be installed in the navigation device that bears the navigation camera. For example, given a technically complex realization additional knowledge or data are then position information about the OP setup, participating personnel, the geometry of the OP room etc.

With regard to the navigation system, the object is achieved by a medical navigation system of the aforementioned embodiment which, according to the invention, thus includes a detection device and a correspondingly operating regulator. The medical navigation system, together with its advantages and the embodiment according to the invention, has already been explained in detail in connection with the method according to the invention.

### BRIEF DESCRIPTION OF THE DRAWINGS

FIG. 1 schematically illustrates a medical navigation system constructed and operating in accordance with the present invention, in an optically operating embodiment.

FIG. 2 schematically illustrates a medical navigation system constructed and operating in accordance with the present invention, in an electromagnetically operating embodiment.

### DESCRIPTION OF THE PREFERRED EMBODIMENTS

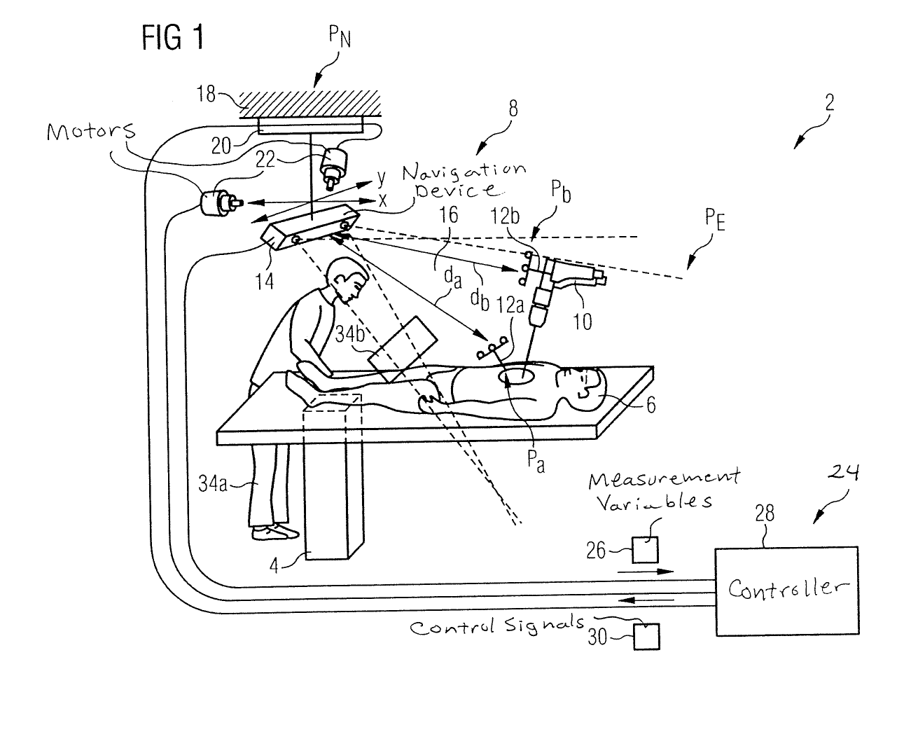

FIG. 1 shows an operating (thus OP) room 2 with a bed 4 on which a patient 6 is borne. An optical navigation system 8 is installed in the OP room 2. A surgical procedure that requires high spatial precision with regard to the procedure location of the instrument 10 at the patient 6 is conducted on the patient 6 with the aid of an instrument 10. The procedure is therefore spatially coordinated with the aid of the navigation system 8. Subjects to be localized are thus the patient 6 and the instrument 10.

The navigation system 8 has two optical markers in the form of the position indicator 12a (which is attached to the patient 6 so as to be stationary) and the position indicator 12b (which is attached to the instrument 10 so as to be stationary). A double camera is used as a navigation device 14 and detects the spatial positions Pₐ and P_{b} of the position indicators 12a and 12b. The navigation device 14 has as an optical detection region (field of view) a navigation region 16 within which the markers 12a and 12b must be kept so that their spatial positions Pₐ and P_{b} can be determined in the navigation system 8.

According to the invention, the navigation device 14 is mounted on the ceiling 18 of the OP room 2 with the use of a rail system 20 that includes two electrical motors 22. With its use the navigation device 14 can be displaced to an arbitrary position P_{N} in the direction of the arrows x and y. The position P_{E} of the navigation region 16 thus can also be displaced. The navigation system 8 moreover has a detection device 24 integrated into the navigation device 14 and the controller 28, which detection device 24 determines both the current attitude or position P_{E} of the detection region 16 in the current position of the navigation device 14, and the positions Pₐ and P_{b} of the position indicators 12a and 12b. In an alternative embodiment, the detection device is a separate module that is installed or can be moved independently of the navigation device 14. In the embodiment shown in FIG. 1, the cameras of the navigation device 14 simultaneously form a portion of the detection device 24 since their images are also used to evaluate or determine position or attitude P_{E} of the navigation region 16. The cameras can moreover be used to observe the navigation region 16 in that they monitor this for the penetration of foreign objects 34a and 34b, for example a person occluding the view of the camera or a voluminous medical apparatus.

The detection unit 24 determines the detected positions Pₐ, P_{b} and P_{E} as measurement variables 26 that are supplied to a regulator 28 that is used by the navigation system 8. From the measurement variables 26 the regulator calculates a control signal 30 to operate the electromotors 22. The controller 28 determines the control signals 30 such that the navigation device 14 is moved to a position P_{N} along the arrows x, y with the aid of the electromotors 22 or the rail system 20, in which position P_{N} the detection region 16 is directed—thus has such an attitude P_{E}—such that the position indicators 12a and 12b lie within this detection region 16. The regulator 28 is an automatically operating controller and regulator. In an alternative embodiment, the cameras in the navigation device 14 can additionally also be rotated and pivoted by additional motors (not shown) in order to be able to correspondingly flexibly align the detection region 16 in suitable positions P_{E}.

In an alternative embodiment, the regulator 28 activates the motors 22 such that the distances dₐ and d_{b} from navigation device 14 to the position indicators 12a and 12b correspond to a predetermined distance, or deviate from this as little as possible or by optimally the same or a maximum amount.

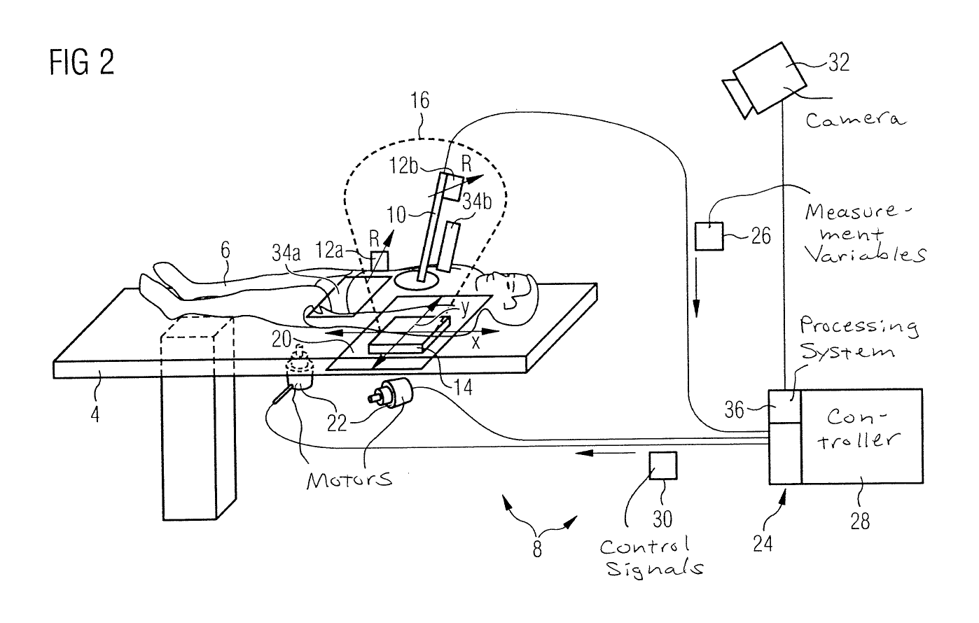

FIG. 2 shows the OP room 2 from FIG. 1 with the patient 6 on whom a different medical measure is conducted with an alternative instrument 10, however. The navigation system 8 in FIG. 2 is an electromagnetic navigation system that uses a field coil as a navigation device 14. Receiver coils as position indicators 12a and 12b are attached both to the instrument 10 and to the patient 6, which receiver coils correspondingly determine the spatial position of patient 6 as position Pₐ and instrument 10 as position P_{b} relative to the navigation device 14.

Here the navigation device 14 is attached via the rail system 20 to the bed 4 and can be displaced along the arrows x and y via motors 22 relative to its spatial position P_{N}. The navigation system 8 includes a controller 28. In this embodiment the detection device 24 is integrated into the controller 28. The detection device 24 again detects the positions Pₐ, P_{b} and P_{E} of navigation region 16 and position indicators 12a and 12b. In the present case, the navigation region 16 is the spatial region surrounding the field coil, in which spatial region this field coil generates a magnetic field that is sufficiently strong and homogeneous for receiver coils. In FIG. 2 the controller 28 also correspondingly receives measurement variables 26 from which it determines the optimal position P_{N} of the navigation device 14 and activates the motors 22 correspondingly in order to move this to the suitable position so that the position indicators 12a and 12b lie securely and optimally in a navigation region 16. In this embodiment the alignment R of the electrical field generated by the field coil is hereby taken into account. The field coil in the form of the navigation device 14 is suitably rotated for this so that the direction R of the field optimally perpendicularly traverses the position indicators 12a and 12b in the form of the receiver coils.

In one embodiment, an additional camera 32 which serves to detect foreign objects 34a and 34b—namely a mounting plate interfering with the navigation region and an ultrasound head—is present as an extension of the detection device 24. Both are metallic and interfere with the field connection between the position indicators 12a and 12b and the navigation device 14. The controller 28 is connected with the camera 32 via an image processing system 36 in order to suitably evaluate the image information and from this to newly calculate the position P_{N} of the navigation device 14 so that the aforementioned interferences are eliminated, meaning that the foreign objects no longer interfere with the navigation region 16 in the relevant region of the position indicators 12a and 12b.

Although modifications and changes may be suggested by those skilled in the art, it is the intention of the inventors to embody within the patent warranted heron all changes and modifications as reasonably and properly come within the scope of their contribution to the art.

We claim as our invention:

1.-13. (canceled)

14. A method for operating a medical navigation system comprising a navigation device having a navigation field of view and at least one position indicator, that is detectable by said navigation device, adapted to be attached to a subject located in said navigation field of view, and a computerized processor, said method comprising the steps of:
with a motorized drive connected to said navigation device, varying a physical location of said navigation device together with said navigation field of view;
with a detection device, detecting a current attitude of said navigation region and said position indicator;
operating said motorized drive from said computerized processor and configuring said computerized processor to embody a regulator coupled with said detection device that operates said motorized drive by supplying a control signal thereto dependent on coordinates of the detected attitude variation of the navigation device, to always maintain said position indicator within said navigation field of view.

15. A method as claimed in claim 14 comprising, via regulator in said computerized processor, regulating said motorized drive to maintain said navigation device at a predetermined distance from said position indicator.

16. A method as claimed in claim 14 wherein said position indicator is a first position indicator, and wherein said navigation system comprises a second position indicator, and comprising, via said regulator in said computerized processor, regulating said motorized drive to maintain said navigation device centered relative to said first and second position indicators.

17. A method as claimed in claim 14 wherein said navigation device comprises optical detectors used by said navigation device to implement navigation, and wherein said method comprises employing said optical detectors of said navigation device to detect said current attitude of said navigation field of view and the position indicator.

18. A method as claimed in claim 14 wherein said navigation device comprises electromagnetic detectors used by said navigation device to implement navigation, and wherein said method comprises employing said electromagnetic detectors of said navigation device to detect said current attitude of said navigation field of view and the position indicator.

19. A method as claimed in claim 18 wherein said optical detectors generate an electromagnetic field, and comprising regulating said motorized drive to cause said electromagnetic field at a location of said position indicator to have a predetermined alignment.

20. A method as claimed in claim 14 comprising providing said computerized processor with a real-time representation of said navigation field of view, and configuring said computerized processor to monitor said navigation field of view to identify interfering foreign objects therein.

21. A method as claimed in claim 20 comprising detecting said current attitude of said navigation device and said position indicator by monitoring said navigation field of view with a camera system that produces a camera image, as said real-time representation of said navigation field of view.

22. A medical navigation system comprising:
a navigation device having a navigation field of view;
at least one position indicator that is detectable by said navigation device, adapted to be attached to a subject located in said navigation field of view;
a motorized drive connected to said navigation device that varies a physical location of said navigation device together with said navigation field of view;
a detection device that detects a current attitude of said navigation region and said position indicator;
a computerized processor that operates said motorized drive from said computerized processor being configured to embody a regulator coupled with said detection device that operates said motorized drive by supplying a control signal thereto dependent on coordinates of the detected attitude variation of the navigation device, to always maintain said position indicator within said navigation field of view.

23. A medical navigation system as claimed in claim 22 wherein said computerized processor is configured to regulate said motorized drive to maintain said navigation device at a predetermined distance from said position indicator.

24. A medical navigation system as claimed in claim 22 wherein said position indicator is a first position indicator, and wherein said navigation system comprises a second position indicator, and wherein said computerized processor is configured to regulate said motorized drive to maintain said navigation device centered relative to said first and second position indicators.

25. A medical navigation system as claimed in claim 22 wherein said navigation device comprises optical detectors used by said navigation device to implement navigation, and said optical detectors of said navigation device also detecting said current attitude of said navigation field of view and the position indicator.

26. A medical navigation system as claimed in claim 22 wherein said navigation device comprises electromagnetic detectors used by said navigation device to implement navigation, and said electromagnetic detectors of said navigation device to detect said current attitude of said navigation field of view and the position indicator.

27. A medical navigation system as claimed in claim 26 wherein said optical detectors generate an electromagnetic field, and wherein said computerized processor is configured to regulate said motorized drive to cause said electromagnetic field at a location of said position indicator to have a predetermined alignment.

28. A medical navigation system as claimed in claim 22 wherein said computerized processor is provided with a real-time representation of said navigation field of view, and wherein said computerized processor is configured to monitor said navigation field of view to identify interfering foreign objects therein.

29. A medical navigation system as claimed in claim 28 comprising a camera that detects said current attitude of said navigation device and said position indicator by monitoring said navigation field of view said camera providing a camera image to said computerized processor, as said real-time representation of said navigation field of view.
